SEQUENCE LISTING (23 sequences)

SEQ ID NO: 1 - protein, 16 aa, Drosophila melanogaster
RQIKIWFQNRRMKWKK

SEQ ID NO: 2 - protein, 7 aa, Artificial Sequence
  WYKYW sequence with GG linker at the N-terminus
GGWYKYW

SEQ ID NO: 3 - protein, 7 aa, Artificial Sequence
  WYKYW sequence with GG linker at the C-terminus
WYKYWGG

SEQ ID NO: 4 - protein, 5 aa, Artificial Sequence
  WYKYW sequence non-mutated
WYKYW

SEQ ID NO: 5 - protein, 5 aa, Artificial Sequence
  WYKYW sequence with alanine mutation
AYKYW

SEQ ID NO: 6 - protein, 5 aa, Artificial Sequence
  WYKYW sequence with alanine mutation
WAKYW

SEQ ID NO: 7 - protein, 5 aa, Artificial Sequence
  WYKYW sequence with alanine mutation
WYAYW

SEQ ID NO: 8 - protein, 5 aa, Artificial Sequence
  WYKYW sequence with alanine mutation
WYKAW

SEQ ID NO: 9 - protein, 5 aa, Artificial Sequence
  WYKYW sequence with alanine mutation
WYKYA

SEQ ID NO: 10 - protein, 5 aa, Artificial Sequence
  WYKYW sequence with D-amino acid mutation
  misc_feature (1)..(1): D-Trp
XYKYW

SEQ ID NO: 11 - protein, 5 aa, Artificial Sequence
  WYKYW sequence with D-amino acid mutation
  misc_feature (2)..(2): D-Tyr
WXKYW

SEQ ID NO: 12 - protein, 5 aa, Artificial Sequence
  WYKYW sequence with D-amino acid mutation
  misc_feature (3)..(3): D-Lys
WYXYW

SEQ ID NO: 13 - protein, 5 aa, Artificial Sequence
  WYKYW sequence with D-amino acid mutation
  misc_feature (4)..(4): D-Tyr
WYKXW

SEQ ID NO: 14 - protein, 5 aa, Artificial Sequence
  WYKYW sequence with D-amino acid mutation
  misc_feature (5)..(5): D-Trp
WYKYX

SEQ ID NO: 15 - protein, 5 aa, Artificial Sequence
  WYKYW sequence with beta3-homo amino acid mutation
  misc_feature (1)..(1): beta3-homo-Trp
XYKYW

SEQ ID NO: 16 - protein, 5 aa, Artificial Sequence
  WYKYW sequence with beta3-homo amino acid mutation
  misc_feature (2)..(2): beta3-homo-Tyr
WXKYW

SEQ ID NO: 17 - protein, 5 aa, Artificial Sequence
  WYKYW sequence with beta3-homo amino acid mutation
  misc_feature (3)..(3): beta3-homo-Lys
WYXYW

SEQ ID NO: 18 - protein, 5 aa, Artificial Sequence
  WYKYW sequence with beta3-homo amino acid mutation
  misc_feature (4)..(4): beta3-homo-Tyr
WYKXW

SEQ ID NO: 19 - protein, 5 aa, Artificial Sequence
  WYKYW sequence with beta3-homo amino acid mutation
  misc_feature (5)..(5): Xaa can be any naturally occurring amino acid
WYKYX

SEQ ID NO: 20 - protein, 5 aa, Artificial Sequence
  WYKYW sequence with beta3-homo amino acid mutation
  misc_feature (2)..(2): beta3-homo-Tyr
  misc_feature (4)..(4): beta3-homo-Tyr
WXKXW

SEQ ID NO: 21 - protein, 5 aa, Artificial Sequence
  WYKYW sequence with beta3-homo amino acid mutation
  misc_feature (1)..(1): beta3-homo-Trp
  misc_feature (2)..(2): beta3-homo-Tyr
XXKYW

SEQ ID NO: 22 - protein, 5 aa, Artificial Sequence
  WYKYW sequence with beta3-homo amino acid mutation
  misc_feature (1)..(1): beta3-homo-Trp
  misc_feature (4)..(4): beta3-homo-Tyr
XYKXW

SEQ ID NO: 23 - protein, 5 aa, Artificial Sequence
  WYKYW sequence with beta3-homo amino acid mutation
  misc_feature (1)..(1): beta3-homo-Trp
  misc_feature (2)..(2): beta3-homo-Tyr
  misc_feature (4)..(4): beta3-homo-Tyr
XXKXW

(19) United States
(12) Patent Application Publication
(10) Pub. No.: US 2022/0389057 A1
(43) Pub. Date: Dec. 8, 2022

(54) ENDOCYTOSIS ROUTING SEQUENCE PEPTIDE FOR CELL DELIVERY SYSTEMS

(71) Applicant: SZEGEDI TUDOMÁNYEGYETEM, Szeged (HU)

(72) Inventors: Norbert IMRE, Kunszentmárton (HU); Tamás MARTINEK, Szeged (HU); Anasztázia HETÉNYI, Szeged (HU); Brigitta BODNÁR, Szeged (HU); Éva MONOSTORI, Szeged (HU); Ágnes CZIBULA, Szeged (HU); Enikö SZABÓ, Ambrózfalva (HU); Mária DELI, Szeged (HU); Péter HORVÁTH, Szeged (HU)

(21) Appl. No.: 17/616,770

(22) PCT Filed: Jun. 8, 2020
(86) PCT No.: PCT/HU2020/050023

§ 371 Date: Dec. 6, 2021

(30) Foreign Application Priority Data
Jun. 7, 2019 (HU) P1900205

(51) Int. Cl. C07K 7/06; A61K 9/127; A61K 9/51

(52) U.S. Cl. CPC C07K 7/06; A61K 9/127; A61K 9/5146; A61K 38/00

(57) ABSTRACT

The present invention relates to cell delivery systems. The present invention specifically relates to new methods of intracellular delivery by endocytosis routing sequence peptides having the sequence of WYKYV or analogues thereof, by caveloar/lipid raft-mediated endocytosis and uses of such peptides. The invention also relates to conjugates comprising said peptides and uses thereof in therapies wherein intracellular delivery of a therapeutically active molecule is required.

### BACKGROUND OF THE INVENTION

### Field of the Invention

The present invention relates to cell delivery systems. The present invention specifically relates to a method of intracellular delivery by endocytosis routing sequence peptides targeting lipid raft-mediated/caveolar endocytosis.

### Description of the Related Art

Mammalian cell membrane is a major obstacle in drug development, because it represents a barrier mostly impermeable for extracellular proteins potentially acting as specific, efficient and tolerable drugs [Fosgerau, K. et al. 2015; Sanchez-Navarro, M. et al., 2017]. Many cell delivery systems have emerged for intracellular delivery, including electroporation, microinjection as physical methods, or by using cationic lipid constructs, protein transduction agents and cell-penetrating peptides [Zuris, J. A. et al., 2015; Erazo-Oliveras, A. et al., 2014; Akishiba, M. et al., 2017, 9, 751] including cyclic [Lattig-Tiinnemann, G et al., 2011; Qian, Z. et al., 2014] and stapled [Walensky, L. D. 2004] derivatives. Despite the swarm of techniques, delivery of macromolecular cargos are rare and only 4% of them are proteins, and very few delivered antibodies. [Gautam, A. et al., 2012]

The closest class of materials related to the field of this invention is the “cell-penetrating peptides”, and we give examples of cell delivery agents and systems, which are described in the following patent applications and patents: WO2014053629A1, WO2016102687A1, CA2971643A1, U.S. Pat. Nos. 8,614,194, 8,409,858, US20170258928. Further, list of known cell penetrating peptides is available in the CPPsite database: http://crdd.osdd.net/raghava/cppsite/[see also: Agrawal P. et al., 2015].

Patents and database entries describe conventional cell penetrating peptides (typically longer than 10 residues) of various compositions including multiple positive charges, hydrophobic side chains with occasionally controlled number of Trp residues. An important attribute of these sequences is their amphipathic nature, which targets the generic features of the mammalian cell membrane, such as the hydrophobic core and the negatively charged surface. Their methods of action lack the high-affinity molecular recognition of a specific native biomolecule on the cell surface, therefore the pathway of translocation is not controlled and is not selective. Since therapeutic protein levels in the extracellular fluid do not exceed the nanomolar range (clinical protocols yield 100-500 nM) [Fischer, S. K. et al., 2012], high-affinity and specific target—carrier interaction is necessary for a cell surface enrichment that facilitates a sufficient material flux in potential clinical applications. Although, some carriers have been shown to translocate membranes with macromolecule cargo, micromolar concentration is required, which prevents their therapeutic applications [Saar, K. et al., 2005] especially when toxicity also enters the picture [El-Andaloussi, S. et al., 2007]. Supercharged protein carriers [McNaughton, B. R. et al., 2009] and their complexes with cationic lipids [Zuris, J. A. et al., 2015] targeting generic negative patches of the membrane can translocate macromolecules through classical endocytosis at nanomolar concentrations.

There are further challenges associated with the endocytotic delivery of therapeutical macromolecules [Zorko, M. et al., 2005], such as the endosomal entrapment which is the fate of the cargo along the classical endocytotic pathways (clathrin-dependent endocytosis and macropinocytosis). Caveolar/lipid raft-mediated (raft-dependent) endocytosis route [Pelkmans, L et al., 2004; Pelkmans, L. et al., 2001] is an exception, and it is also exploited by viruses (simian virus 40 and murine polyoma virus) [Smith, A. E. et al., 2004], bacterial toxins (cholera and tetanus) [Montesano, R., et al., 1982], and endogenous proteins [Fajka-Boja, R., et al., 2008] to deliver the macromolecules in their functional form without endosomal entrapment and degradation.

Fajka-Boja, R., et al. [Fajka-Boja, R., et al., 2008] teach, giving a summary of the prior art knowledge, that Gal-1 probably binds to the raft-component, GM1 ganglioside, whereby Gal-1 binding results in raft formation in T cell membrane. The plausible mechanism is that Gal-1 induces rapid reorganization of the lipid microdomains, then is delivered as a cargo to endosomes and the Golgi system. The inhibition of the raft-dependent pathway did not reduce either the internalization of Gal-1 or GM1, whereas blocking clathrin-mediated endocytosis along with disruption of lipid rafts leads to the complete arrest of Gal-1 internalization. Therefore, Gal-1 applies more than one pathways to enter into Jurkat cells, and entry of Gal-1 into the cell is mediated by GM-1 via a clathrin-mediated pathway. Fajka-Boja, R., et al. does not provide any hint on finding or using an endocytosis routing sequence.

The entry through ganglioside recognition, by triggering caveolar endocytosis, is an attracting strategy, because the progression of the internalized caveolae to endosomes and later to lysosomes is relatively slow or absent allowing time for delivery to cell organelles and cytosol [Kiss, A. L. et al., 2009; Matsubara, T., et al., 2017; De Jong, E., et al., 2018]. However, while avoiding endosomal entrapment and lysosomal degradation is a crucial step, designing cell-penetrating and useful internalizing peptides meeting this aim is a grand challenge, in spite of serious efforts made towards designing new strategies to prevent degradation [LeCher, J. C. et al., 2017]. The molecular level understanding of endocytosis pathways is still in development, therefore it is currently unpredictable if a successful internalization avoids lysosomal degradation. It is not obvious whether a route inside the cell protects the cargo from degradation, and whether the internalized cargo will stay functionally intact.

Moreover, high-affinity (low nanomolar) molecular recognition (necessary for therapeutical applications) is currently an unsolved problem. Construction of high affinity glycolipid binding peptide sequences has still been a great unmet fundamental challenge before our results, because these compounds do not form binding cavities/hotspots accomodating peptidic segments or side chains Relatively large peptides (15mers) in the prior art [Landon, L. A. et al., 2004; Matsubara T. et al., 1999; Matsubara T. et al., 2007] displayed affinities in the μM range only, which reflects insufficient affinity binding in terms of medical uses.

The specific and high-affinity targeting of ganglioside GM1 is especially sought, because it is expressed in many mammalian cell types including endothelial cells and overexpressed in cancer cells [Fuster, M. M. et al., 2005; Krengel, U. et al., 2014, 5, 325], a feature which could be used in targeted therapy, e.g. cancer therapy.

The present inventors have serendipitously found a pentameric sequence which recognizes cerebroside GM1 with an unexpected and unprecendented affinity and selectivity. WYKYW, named by the use of the one-letter symbols and as Trp-Tyr-Lys-Tyr-Trp by the use of the three-letter symbols applied in this file (https://www.tocris.com/resources/peptide-nomenclature-guide)—was first identified by Gabius and coworkers [André, S. et al., 2007; Maljaars, C. E. P., et al., 2008] as a member of the Tyr-Xxx-Tyr containing pentapeptide family, which were observed to decouple interactions between galectin-1 (Gal-1) and the proteoglycan asialo-fetuin (glycomimetic) at high (micromolar) concentrations [Andre, S., et al., 2005], i.e. as a potential week asialofetuin (glycoprotein) binding segment [Weber, E. et al., 2010]. However, the previous art was silent about binding of WYKYW to the cerebroside GM1, and does not provide any hint on the high affinity and specificity of this binding. The glycoprotein asialofetuin is a fundamentally different class of material than the ganglioside (cerebroside) GM1.

The research group of the inventors showed in earlier experiments that the mechanism of decoupling of the Gal-1—asialo-fetuin interaction is based on the competitive binding of the peptide at the glycan moiety [Weber, E. et al., 2010]. From the fact, however, that Gal-1 binds weakly to ganglioside GM1 [Kopitz, J. et al., 1998], it could not be predicted/foreseen that our lead sequence interact strongly and selectively with ganglioside GM1.

The structure-activity relationship studies revealed that small changes can alter the affinity dramatically, hence the GM1 binding of the WYKYW peptide is highly selective, which has been impossible to predict at the state-of-the-art level of understanding of molecular recognition. The high affintiy interaction between WYKYW and GM1 could not be foreseen in the prior art.

Based on the above finding the present invention defines a novel class of materials capable of routing the cargo to the entrapment-free caveolar/lipid raft-mediated endocytosis at nanomolar (therapeutically relevant) concentrations by high affinity to and specific recognition of molecular receptors involved in the desired endocytosis pathway. We denote these materials as “endocytosis routing sequences” (ERS).

It is well known in the art that lipid raft mediated endocytosis via GM1 requires clustering of GM1 at the membrane surface with multivalent ligands [Pelkmans, L. et al., 2002]. To the contrary, the present inventors have found that the stoichiometry of the low nanomolar WYKYW-GM1 interaction is 2:1, which is against any clustering mechanism. Moreover, the present inventors have shown that already a single copy of the WYKYW segment on the cargo is sufficient to induce endocytosis, again pointing to the absence of GM1 clustering. It is therefore an unexpected and unpredictable result that the lipid raft-mediated endocytosis can be induced with the WYKYW sequence. Therefore, the present invention discloses a novel process of clustering-free WYKYW induced endocytosis.

While there are many known cell-penetrating peptides, only 63% of them can internalize dyes, and 33% of them can internalize small molecules. Delivering larger, potentially useful cargoes have been only achieved in 4% of all the sequences known in the prior art [Kauffman, W. B. et al. 2015]. Even when a cell-penetrating peptide is reported to internalize larger cargoes, it heavily depends on the specific cargo molecule and the extracellular concentration. Many archetype cell-penetrating peptides (such as penetratin, used in our control measurements) fail to deliver larger cargo at concentrations below the micromolar range. The present inventors have unexpectedly found that the peptides according to the invention can internalize a large cargo, even as large as an IgG complex which has not been achieved with any known or newly designed cell-penetrating peptide, especially not with one comprising a minimal pentameric peptide motif [Kauffman, W. B. et al. 2015].

Moreover, quite unexpectedly, the present inventors have found that the sequences of the invention induce a low degradation or degradation free endocytosis of cargos which are protected inside the cell.

Thus, the present invention has one or more of the following effects

(i) an unexpectedly high-affinity and specific interaction of sequence WYKYW and the close analogues thereof (which are also “endocytosis routing sequences”, i.e. ERSs) with ganglioside GM1 facilitating the endocytosis routing by enrichment on GM1 receptors,

(ii) ability of WYKYW to specifically trigger the caveolar/lipid raft-mediated endocytotic process at low nanomolar concentrations,

(iii) ability of WYKYW to act as a carrier for cargos from small molecules up to associates of macromolecules,

(iv) a low level of degradation of the cargos in the cells after endocytosis.

These features lead to a novel and efficient cell delivery system based on ERSs according to the present invention. Thus the cell delivery system according to the present invention substantially departs from the conventional concepts and designs of the prior art. Accordingly, it provides a process primarily developed for the purpose of delivering therapeutic or diagnostic molecules, biomolecules, macromolecules and the associates thereof to the site of action in the cells in a bioactive form at extracellular concentrations relevant in medical or research applications.

### SUMMARY OF THE INVENTION

Accordingly, the invention relates to the following aspects:

1. A non-therapeutic use of a peptide of general formula R1-R2-Lys-R3-Trp,

wherein R1 is Trp or β³-homo-Trp, R2 is Tyr or β³-homo-Tyr, and R3 is Tyr or β³-homo-Tyr,

as an endocytosis routing sequence peptide (ERS peptide) for mediating the delivery of a cargo into cells. Said delivery preferably occurs via ganglioside binding triggering lipid-raft mediated endocytosis.

2. The use according to point 1, wherein said cargo is selected from the group consisting of biologically active molecules, diagnostic molecules and nanoparticles, wherein preferably the biologically active molecules are polypeptides or comprise polypeptides as a biologically active moiety; and preferably the diagnostic molecules are polypeptides or comprise polypeptides. Preferably the diagnostic molecules comprise a label which render said diagnostic molecules detectable. Said nanoparticles preferably include or comprise liposomes and/or polimersomes. Said nanoparticle also can be used for the delivery of small molecules (non-polymers and/or not macromolecules).

3. The use according to point 1 or 2, wherein the peptide has the sequence of Trp-Tyr-Lys-Tyr-Trp. The peptide may be in the form selected from the group consisting of its salts, amides, protected forms and its conjugates.

4. A conjugate of a peptide of general formula R1-R2-Lys-R3-Trp, wherein R1 is Trp or β³-homo-Trp; R2 is Tyr or β³-homo-Tyr, and R3 is Tyr or β³-homo-Tyr, said conjugate comprising

said peptide as an endocytosis routing sequence peptide (ERS peptide),

a cargo, and

optionally a linker moiety between said peptide and cargo.

The peptide in the conjugate may be in the form selected from the group consisting of its salts, amides and protected forms.

The cargo may be a molecule (cargo molecule) or a combination or cluster of molecules or a substance having the size suitable for delivery into the cell, e.g. a nanoparticle. In an embodiment the cargo molecule can be nucleic acid, a peptide, a protein, a lipid, and/or an oligo or polysaccharide or a combination thereof including lipoproteins and glycolipids.

5. The conjugate according to point 4 wherein said cargo is selected from the group containing biologically active molecules, diagnostic molecules and nanoparticles.

Biologically active molecules, delivered into the cells as cargos, are molecules which are capable of eliciting or inducing a biological effect within the cell into which the cargo is delivered. Preferred biologically active molecules have a beneficial effect on the cell, and preferably on a tissue which comprises said cell. In a preferred embodiment the biologically active molecule has a beneficial effect on the organism which comprises said cell or tissue. Preferably the biologically active molecules are pharmaceutically active agents, e.g. medicines.

Diagnostic molecules are cargo molecules which, if present, provide or result in a signal and thereby information about the condition or status of the cell into which they are delivered, or about the tissue comprising said cell or about the organism comprising said tissue. Diagnostic molecules optionally comprise a label which render said diagnostic molecules detectable.

Nanoparticles are a nano-size objects with all three external dimensions in the nanoscale, wherein at least one dimension is in the 1 and 100 nanometres (nm) range or two all three dimensions thereof is in this range. Typically longest and shortest axes of nanoparticles do not differ significantly, in particular a significant difference typically being a factor of at least 3. Preferably the nanoparticle has a surrounding interfacial layer. This can be e.g. a surface coating which may comprise biologically active molecules or diagnostic molecules. Moreover, the nanoparticles may be hollow (may comprise an internal space or cavity) and the layer may provide a barrier between the internal space and environment. Thus, the layer may comprise amphiphilic molecules like lipids or polymers and preferably may form a membrane. Thus, preferably the nanoparticles are liposomes or vesicles or polymersomes. Thus, said nanoparticle may comprise biologically active molecules or diagnostic molecules in their interior space or in their membrane.

Moreover, nanoparticles may comprise small molecules (non-polymers and/or not macromolecules).

6. The conjugate according to point 4 or 5, where the ERS peptide and the biologically active molecule are bound together via a linker, preferably coupled to the N-terminus of the said endocytosis routing sequence, which is selected form the group containing:

a) a spacer sequence, preferably an organic oligomer, e.g. an oligopeptide,

b) a stabilizing moiety or sequence, preferably an oligopeptide of positive charge,

c) an organic, water-soluble, biologically compatible polymer,

d) any combination of linkers a) to c).

Preferably, in the conjugate the biologically active molecule are bound together via a linker, which is selected from the group containing:

a) an oligopeptide, preferably an oligopeptide comprising A and/or G, more preferably a GG,

b) an oligopeptide of positive charge having a length of at least 7 or at least 9 amino acids, more preferably a penetratin moiety (RQIKIWFQNRRMKWKK) or analogue thereof coupled preferably to the N-terminus of the said endocytosis routing sequence,

c) a PEG-based oligomeric moiety, preferably the oligomeric moiety comprising 1, 2, 3 or 4 of amino-PEG2-ethyl-succinamic acid parts, coupled consecutively, preferably to the N-terminus of the said ERS peptide and

d) any combination of linkers a) to c).

7. The conjugate according to any of points 4 to 6, where the ERS peptide has the sequence of Trp-Tyr-Lys-Tyr-Trp.

8. The conjugate according any of points 4 to 7 wherein the linker or the stabilizing moiety or sequence is the penetratin molecule (RQIKIWFQNRRMKWKK).

9. A polynucleotide encoding a polypeptide suitable for mediating the delivery of a protien cargo into cells via ganglioside binding triggering lipid-raft mediated endocytosis, said polypeptide comprising

a peptide having the sequence of Trp-Tyr-Lys-Tyr-Trp, and

a linker moiety, and/or

a cargo molecule.

10. The conjugate according to any of points 4 to 8 or the polynucleotide according to point 9 for use in the therapy of a disease wherein the cargo is a therapeutically active molecule.

Preferably the disease is selected from a condition wherein the patient cells into which the cargo is to be delivered are to be killed. Such cell may be cancer cells or cells infected by a pathogenic agent.

In an embodiment the therapy is directed to a condition wherein the cells of the patient express ganglioside GM1, preferably overexpress GM1. In a preferred embodiment the condition is cancer and the therapeutically active molecule is an anti-cancer molecule.

Preferably the disease is selected from a condition wherein the patient cells into which the cargo is to be delivered are to be revived or healed.

In a preferred embodiment the therapy include a treatment selected from the group consisting of (i) protein restoration, (ii) inhibition of intracellular protein—protein interactions with antibodies, proteins or protein mimetic inhibitors, (iii) gene therapy and (iv) chemotherapy.

In particular in the therapy low systemic drug concentration is needed and crossing the cell membrane is essential.

11. In the above embodiments the sequence of said peptide is particluarly selected from

Trp-Tyr-Lys-Tyr-Trp, or its analogues selected from

Trp-Tyr-Lys-β³-homo-Tyr-Trp,

Trp-β³-homo-Tyr-Lys-Tyr-Trp,

Trp-β³-homo-Tyr-Lys-β³-homo-Tyr-Trp,

β³-homo-Trp-Tyr-Lys-Tyr-Trp,

β³-homo-Trp-β³-homo-Tyr-Lys-Tyr-Trp,

β³-homo-Trp-β³-homo-Tyr-Lys-β³-homo-Tyr-Trp,

β³-homo-Trp-Tyr-Lys-β³-homo-Tyr-Trp.

Preferably, the sequence of said peptide or its analogue is selected from

Trp-Tyr-Lys-Tyr-Trp,

Trp-Tyr-Lys-β³-homo-Tyr-Trp,

Trp-β³-homo-Tyr-Lys-Tyr-Trp,

β³-homo-Trp-Tyr-Lys-Tyr-Trp.

An embodiment of the invention relates to these peptide analogues of Trp-Tyr-Lys-Tyr-Trp or a peptide analogue as defined in points 1 above or here in point 11. The peptide analogues may be in the form selected from the group consisting of its salts, amides, protected forms and a part of conjugates.

12. In an embodiment the invention relates to a pharmaceutical composition for use in a therapy.

13. In an embodiment the invention relates to a therapeutic method or a method for treatment said method comprising administration of the conjugate of the invention in an effective amount to a patient in need of a therapeutic molecule, said conjugate comprising the therapeutic molecule as a cargo.

In a preferred embodiment the therapy is the therapy of a disease wherein said subject has said disease and wherein the cargo is a therapeutically active molecule.

Preferably the disease is selected from a condition wherein the patient cells into which the cargo is to be delivered are to be killed. Such cell may be cancer cells or cells infected by a pathogenic agent.

In an embodiment the therapy is directed to a condition wherein the cells of the patient express ganglioside GM1, preferably overexpress GM1. In a preferred embodiment the condition is cancer and the therapeutically active molecule is an anti-cancer molecule.

Preferably the disease is selected from a condition wherein the patient cells into which the cargo is to be delivered are to be revived or healed.

In a preferred embodiment the therapy include a treatment selected from the group consisting of (i) protein restoration, (ii) inhibition of intracellular protein—protein interactions with antibodies, proteins or protein mimetic inhibitors, (iii) gene therapy and (iv) chemotherapy.

In particular in the therapy low systemic drug concentration is needed and crossing the cell membrane is essential.

### BRIEF DESCRIPTION OF THE FIGURES

FIG. 1 Structures of the targeted gangliosides, peptides and binding data

(a) Structures of the targeted ganglioside GM1 and its truncated derivatives.

(b) Sequence and structure of lead peptide sequence 1 (WYKYW). Binding affinities (Kd) and stoichiometries (n) for ganglioside—WYKYW interactions measured by isothermal titration calorimetry ITC. Parameters were obtained with non-linear least squares fitting against the two independent- and the single binding site models for GM1 and GM3, respectively. For asialo-GM1, ITC enthalpogram did not exhibit finable features.

FIG. 2. Cellular uptake of fluorescently labelled peptides and their cytotoxicity

(a) and (b) Uptake of carboxyfluorescein (CFU) labeled peptides (CFU-WYKYW, CFU-Penetratin, and CFU-Penetratin-GG-WYKYW) at 1 μM after 1 hour in (a) HeLa and (b) Jurkat cells.

(c) Cytotoxicity of CFU-Penetratin-WYKYW and Biotin-Penetratin-GG-WYKYW at 10 μM after 24 hours on HeLa cell as determined by bioimpedance measurements. Triton X-100 (TX) was used as toxicity control. All data points depict a mean of three independent measurements and error bars show the standard error of mean.

FIG. 3. Representation of the (ERS-linker-biotin)₄NeutrAvidin complex

Schematic representation of the. The linker can be an oligomeric peptide sequence, for example a Penetratin-GG oligomeric peptide sequence (WYKYW-GG-Penetratin-biotin)₄NeutrAvidin complex, or another type of linker, e.g. a PEG-based oligomeric linker ((WYKYW-PEG-biotin)₄NeutrAvidin complex) attached to the N-terminal of the WYKYW ERS peptide sequence (see “ERS” on the Figure).

FIG. 4. Cellular uptake and mechanims of FITC-NeutrAvidin containing complexes

(a) Internalization for Biotin-Penetratin and Biotin-Penetratin-GG-WYKYW as well as Biotin-PEG-WYKYW complexed with FITC-NeutrAvidin at 1 μM in HeLa cells after 1 hour as determined by flow cytometry.

(b) Influence of endocytosis inhibitors on cellular uptake. HeLa cells were preincubated with inhibitors wortmannin, chlorpromazine, β-methyl-cyclodextrin at 37° C. for 30 or 60 min, and subsequently incubated with Biotin-Penetratin-GG-WYKYW complexed with FITC-NeutrAvidin at 37° C. for 60 min. Control experiment was also performed at 4° C. All data points depict a mean of three independent measurements and error bars show the standard error of mean.

(c) Live confocal laser scanning microscope images of HeLa cells incubated with Biotin-Penetratin, Biotin-Penetratin-GG-WYKYW and Biotin-PEG-WYKYW, complexed with green fluorescent FITC-NeutrAvidin (light grey dots in the cytosol; originally green) at different concentrations after 1 hour. Hoechst 33342 stained nuclei of the HeLa cells in cyan (oval shape nuclei, light grey), and LysoTracker Red stained lysosomes in magenta (larger dark grey spots in the cytosol when Penetratin was part of the complex). No lysosomes are stained when conjugates with the PEG-based linker was applied.

FIG. 5. Representation of complex with IgG containing cargo

The labeled IgG containing complex of the invention is exemplified by the schematic representation of

the (WYKYW-GG-Penetratin-biotin)₃NeutrAvidin-biotin-IgG-IgG-r-Phycoerythrin (RPE) complex.

FIG. 6. Cellular targeting with the IgG containing complex

(a) Artificial intelligence-aided quantitative analysis of the live CLSM images. HeLa cells were incubated for 3 hours with different concentrations of the IgG complex, comprising NeutrAvidin hub protein, Biotin-Penetratin-WYKYW carrier, a biotinylated IgG antibody, and its secondary antibody conjugated with r-phycoerythrin (total molecular weight: 580 kDa) then at least 75 representative cells were analyzed at each concentration.

(b) Delivery of the IgG complex into HeLa cells at different concentrations after 3 hours. Hoechst 33342 stained nuclei of the HeLa cell (indicated in light grey oval shape nuclei, originally cyan), WGA-FITC staining defines cell membranes (indicated in light grey dotted lines, originally green), r-phycoerythrin (RPE) is indicated in dark grey in the cytosol (originally: magenta), in cells treated with complex at 20 nM (lower level of RPE) and 40 nM (higher levels of RPE). Control cells were treated with RPE labelled secondary antibody at 160 nM for 3 hours.

### DETAILED DESCRIPTION OF THE INVENTION

In view of the foregoing disadvantages being inherent in the known types of cell delivery systems by the present invention a new cell delivery method is provided, which can be utilized for specific delivery of different “cargos”, e.g. therapeutic or diagnostic molecular, biomolecular, macromolecular species and the associates thereof by routing to lipid raft-mediated/caveolar endocytosis at extracellular concentrations relevant from therapeutical and diagnostic aspects. To attain this, the present invention generally applies a high-affinity molecular recognition event between ganglioside GM1 and a short oligomeric peptide, that is an endocytosis routing sequence peptide (ERS peptide), which interaction triggers the lipid raft-mediated/caveolar endocytosis at low nanomolar concentrations. The present invention provides a class of easily applicable, non-toxic, efficient short carrier peptide tags for intracellular delivery.

The ERS peptides are able to translocate said cargos through the cell membrane in their intact and bioactive form to the intracellular site of action, preferably via endocytosis. The ERS peptides are cell penetrating peptides. The translocation is carried out through the specific binding of the ERS peptides of the invention to ganglioside GM1, an ambundant component of the eucaryotic cell membrane, which binding triggers lipid-raft mediated endocytosis. This new cell delivery method based on the use of the specific ERS peptide tags is not anticipated, rendered obvious, suggested, or even implied by any of the prior art, either alone or in any combination thereof since no hint can be found in them for such activity for our lead peptide of Trp-Tyr-Lys-Tyr-Trp (WYKYW).

Thus, ERS peptide WYKYW and its high affinity derivatives do not only bind GM1 but also trigger the process of lipid raft-mediated/caveolar endocytosis in human cells.

The term “peptide” is understood herein as sequence of amino acids linked by amide bonds. Preferably the term includes herein the complete ERS peptide and any residues or moieties having the same amino acid sequence. The ERS peptide may form part of a larger peptide and in this case the ERS peptide part is called herein as ERS peptide.

An aspect of the invention is that the ERS peptide Trp-Tyr-Lys-Tyr-Trp (WYKYW) and its high affinity derivatives in the use/method according to the invention binds ganglioside GM1 with a dissociation constant (Kd) of less than 200 nM. This high affinity and selective interaction is essential for routing the cargo selectively to the GM1-containing lipid rafts without enrichment on the cell surface at other unwanted entry points (clathrin-dependent endocytosis or macropinocytosis) and allowing enrichment at lipid rafts at therapeutically relevant concentrations.

The invention also relates to WYKYW analogues which are selected from the group of backbone homologues obtained by α->homo-β³ amino acid mutations, in particular from the group consisting of bWYKYW, WbYKYW and WYKbYW which have affinities to GM1 below 200 nM. These β-mutations improve the peptidase/protease resistance at the same time.

A “homo-β³ amino acid” as used herein is an amino acid, either as a molecule or as a part of an amino acid sequence, i.e. an amino acid residue, which comprises, if derived from an alpha amino acid, an inserted CH₂ group between the alpha carbon atom and the carbonyl group. A “homo-β³ amino acid” residue has the following structure of formula (1), if it forms part of a polypeptide chain.

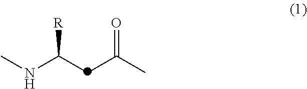

or

the formula of a polypeptide consisting of homo-β³ amino acids is shown as formula (2):

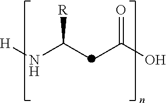

wherein R is a side chain of protein forming amino acid, preferably tryptophane or tyrosine, herein, and represents the inserted CH₂ group.

The ERS-peptide may be utilized as a separate molecule for the preparation of a conjugate comprising at least the ERS-peptide, a linker and a cargo for the routing of the cargo into a cell.

The peptide is preferably used in the form of or as a part of a conjugate, wherein said conjugate comprises at least the ERS peptide and the cargo to be introduced into the cell via ganglioside binding triggering lipid-raft mediated endocytosis.

The conjugate preferably also comprises a linker moiety attached via covalent bond to the N terminus of the ERS peptide. The linker preferably provides connection between the cargo and the ERS peptide.

The linker may have several functions.

The linker may be a spacer sequence or may have a spacer function in the conjugate. A spacer is considered to have the role of providing spatial exposure of the ERS peptide so that the binding to the GM1 or analogue may not be sterically hindered. The present inventors have found that if carboxyfluorescein is attached to the ERS peptide (like WYKYW) without a spacer, the binding affinity to the GM1 has been significantly reduced (see Table 2).

The spacer sequence may comprise or may be an oligopeptide, e.g. an oligomer of small apolar amino acid like G (Gly), A (Ala) or I (Ile) or a sequence comprising a combination thereof, preferably G or A. More preferably the spacer comprises an oligomer of G, highly preferably the spacer is GG.

The present inventors have found that the linker also may be a PEG-based oligomer chain, such as a trimer of (8-amino-3,6-dioxa-octyl)succinamic acid. The PEG-based linker also has the role of a spacer. Moreover, as it is known from the art [Knowles, D B et al., 2015] the PEG also has a stabilizing effect on proteins, and thus may stabilize the cargo in the present invention.

A PEG-based oligomer chain or a PEG-based oligomeric moiety (used interchangeably herein) is understood herein as a chain or moiety comprising at least adjacent (contiguous) ethylene-oxide monomer units. A linker comprising such moiety may be named herein as a PEG-based linker. Preferably the PEG-based oligomeric moiety comprises 2 to 20, more preferably 4 to 10 ethylene-oxide monomer units. In an embodiment the PEG-based oligomeric moiety consists of ethylene-oxide monomer units (PEG oligomer or PEG oligomeric moiety). In an other embodiment the PEG-based oligomeric moiety is a co-oligomer and also comprises other unites, e.g. ethylene-amide residues. In the exemplary conjugates of the invention the PEG-based oligomeric moiety is denoted as “PEG” for sake of brevity. In a preferred embodiment the PEG-based oligomeric moiety comprises 1, 2, 3 or 4 of amino-PEG2-ethyl-succinamic acid parts, coupled consecutively to the N-terminus of the said ERS peptide, wherein PEG2 stands for two ethylene-oxide monomer units (in line with the usual nomenclature applied in the field of invention).

Thus, it follows that in a preferred embodiment the linker has a stabilizing role on the cargo, in particular on polypeptide or protein-type or—containing cargos, or the linker comprises such a stabilizing element.

It has been also found that a penetratin sequence can be used as a linker, where it may have a stabilizing effect on the conjugate without any direct influence on the endocytosis of the protein cargo. This effect preferably takes place at the concentration of 1 μM or below. The solubilisation is typically achieved by an extra charge density of the penetratin sequence. In particular, IgG conjugates comprising a penetratin linker proved to be soluble enough to undergo endocytosis, while PEG-based linker induced precipitation under the same circumstances.

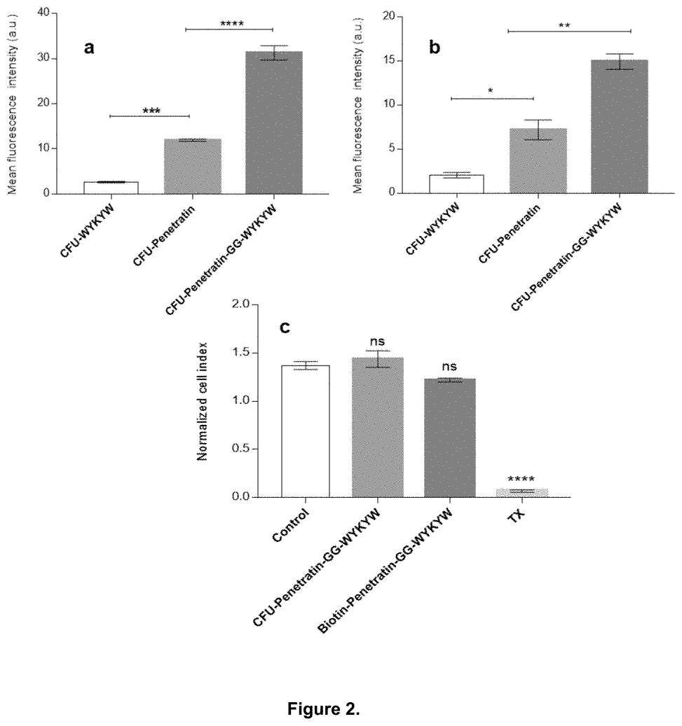

Without protein cargo, sequence WYKYW or its analogue attached to Penetratin increases the cell penetration efficiency of Penetratin at 1 μM (FIG. 2). This concentration is a magnitude below at which concentration Penetratin is normally applied (10 μM, HeLa and Jurkat cells) because it opens up a new entry point for Penetratin and improves cell surface enrichment.

Penetratin is a 16-amino-acid sequence, RQIKIWFQNRRMKWKK. In the present invention the linker optionally may comprise, as a stabilizing sequence, a penetratin fragment. The penetratin fragment is at least 7, preferably at least 9, more preferably at least 13 amino acid long.

Furthermore, an analogue of the penetratin or penetratin fragment can be used. The analogue may have the a sequence which is identical with the corresponding penetratin sequence segment or which shows at least 70%, 80% or 90% therewith or which is different in at most 5 amino acids, preferably at most 4 or at most 3 amino acids in the case of an at least 13 amino acid long analogue or which is different in at most 2 or 3 amino acids in case of an at least 9 amino acid long analogue or which is different in at most 2 amino acids in case of an at least 7 amino acid long analogue. However, preferably, the number of positively charged residues are maintained in the analogue if aligned with the corresponding penetratin sequence.

Without being bound by theory, it is also contemplated that other oligopeptide linkers which have a beneficial effect on the stabilization of cargos are useful in the present invention.

The cargo can be attached (bound or linked) to the ERS-peptide preferably via a linker moiety.

In an embodiment the cargo is attached to the linker moiety via covalent linkage or bond.

In a preferred embodiment the cargo is derivatized and linked or bound to the linker via a spacer.

In a further embodiment the cargo is attached to the linker moiety via a ligand-receptor interaction. A ligand-receptor interaction as understood herein comprises a binding molecule or receptor and a ligand which is bound by said binding molecule with a high affinity sufficiently strong to provide that the cargo, or a sufficiently high ration thereof, is not dissociated from the ERS-peptide (or linker plus ERS-peptide) part upon routing into the cell.

The receptor is or comprises typically a protein molecule which is capable of binding a ligand of strong affinity. A ligand may be for example another protein or peptide molecule or an organic molecule which can be bound or linked to the cargo or to the ERS-peptide (or preferably to the linker). Either the binding molecule or the ligand can be bound to the cargo or to the ERS-peptide (or preferably to the linker). Preferably the binding molecule is bound to the cargo and the ligand is bound to the linker.

In an example the binding molecule is a kind of avidin, preferably NeutrAvidin and the ligand is biotin. The skilled person will understand that other examples may be applied. A similar affinity-based linkage can be the Calmodulin binding peptide-Calmodulin interaction [Journal of Cell Science (2016) 129, 2473-2474].

In an embodiment the the binding molecule is considered as the cargo.

In a further embodiment the binding molecule has more than one binding site. Within this embodiment the cargo can be attached to the binding molecule via a ligand to one or more of the binding sites of the binding molecule, whereas the ERS-peptide (preferably via a linker and ligand) can be attached to the binding molecule via other one or more binding site(s). An example for this embodiment is the NeutrAvidin plus biotin complex wherein biotin can be bound to an ERS-peptide plus ligand moiety which is thus capable of binding to a binding site of NeutrAvidin whereas the cargo can be linked, optionall via a linker to a biotin to form a conjugate which is thus also capable of binding to the binding protein NeutrAvidin.

In the terminology used herein both the ligand-linker-ERS-peptide moiety or molecule and the cargo-ligand or cargo-linker-ligand moiety or molecule is called herein a conjugate, whereas the whole complex comprising the conjugates and the binding molecule is also covered by the term conjugate.

Thus, the conjugate of the present invention may comprise a linker which optionally has a role of a spacer and optionally has a role of stabilizer. Preferably the linker comprises a spacer moiety, like a spacer peptide and a stabilizer moiety. The stabilizer moiety may be in a preferred embodiment a cationic or arginine-rich peptide In a particular embodiment in case of large protein cargos, like immunoglobulins or immunoglobulin fragments, such cationic or arginine-rich peptide linkers are applied in the conjugate according to the present invention to stabilize the system in the solution phase. In a further preferred embodiment the conjugate of the present invention is formed by providing such a conjugate comprising the ERS-peptide, a linker and a ligand and by providing a further conjugate of a cargo and a ligand or optionally a cargo and a ligand connected via a linker, and one or more of each conjugates are attached to a binding molecule (considered as receptor, preferably by a receptor-ligand binding). In this case the binding molecule having multiple binding sites may serve as a hub for binding one or more cargo-carrying conjugate(s) and one or more ERS-peptide containing conjugate(s).

In a set of variant embodiments, the binding molecule—ligand attachment may be replaced by or modified to a covalent binding by derivatization and chemically binding of the binding partners.

The present invention is useful for routing essentially any cargo, as defined herein, to the cytosol via ganglioside binding triggering lipid-raft mediated endocytosis which may escape lysosomes.

In a preferred embodiment the cargo is a therapeutic molecule. In a preferred embodiment the cargo is a therapeutic protein or polypeptide.

An example for large protein cargos is an immunoglobulin or an immunoglobulin fragment. The immunoglobulin or an immunoglobulin fragment can be engineered. The immunoglobulin or an immunoglobulin fragment can be a monoclonal antibody or a fragment thereof.

The invention also relates to a composition, preferably a pharmaceutical composition according to the invention.

A “composition” as used herein is a composition of matter which comprises a conjugate according to the invention in an amount effective for the composition is to be used for and and at least one type of further substance. Compositions may also comprise further biologically active substances useful e.g. in a combination therapy. Preferably the compositions comprise one or more biologically inactive substance(s), e.g. biologically acceptable carriers, formulation agents, excipients etc or the active substance is present in a matrix. which additional substances are well known in the art.

Examples for combination therapy include further protein restoring agents, protein-protein interaction inhibitors, agents or supplementary agents for gene therapy as well as anti-cancer agents.

A pharmaceutical composition is a composition for use in therapy.

A “therapy” refers to any process, action, treatment method or the like, wherein the subject or patient is under aid, in particular medical or veterinarian aid with the object of improving the subjects's or patient's condition including restoring healthy condition, either directly or indirectly; preferably the administration of the conjugate of the invention in an effective amount. Therapy includes both treatment of a disease condition which has been developed and prevention of said condition to develop or to worsen in a subject or patient. The terms “effective amount” qualifies the amount of a compound required to relieve or prevent (or prevent worsening of) one or more of the symptoms or characteristic parameters of a condition, e.g. disease or disorder. In other words wherein it exerts a therapeutic effect in the therapy. In particular “effective amount” relates to the amount which is capable of delivering, by endocytotic delivery, the conjugate of the invention into cells of a subject or patient in an amount wherein the cargo, which is preferably a therapeutical macromolecule, in a level wherein said cargo exerts a therapeutic effect in the subject or patient.

Throughout this specification, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. The term “essentially consist of” is a particular embodiment of the term “comprise”, wherein any other essential non-stated member, integer or step, i.e. which has the same use or effect as the stated member, is excluded.

In the context of the present invention, the term “comprise” encompasses the term “essentially consist of” which in turn encompasses the term “consist of” and each can be amended to the term encompassed.

The terms “a” and “an” and “the” and similar reference used in the context of describing the invention (especially in the context of the claims) are to be construed to cover both the singular and the plural, unless otherwise indicated herein or clearly contradicted by context.

Fields that could benefit from the use of the present invention related to ERS peptides are (i) protein restoration, (ii) inhibition of intracellular protein—protein interactions with antibodies, proteins or protein mimetic inhibitors, (iii) gene therapy and (iv) cancer therapy or (v) therapy in case of a pathogenic infection. In these cases reaching intracellular targets at low systemic drug concentration is needed and crossing the cell membrane is the major pharmacokinetical bottleneck in the efficacy. Thus, the invention also relates to a therapeutic method comprising a treatment of a patient in need of any of these treatment methods or steps.

Protein restoration treatment (i) aims at restoring or providing protein function in a cell and/or restoring an impaired function, and may include administration of a protein in which the cell is depleted or an agent, e.g. protein which restores the activity of the one impaired, e.g. a chaperon or the like. In another approach such therapy may include administration of a nucleic acid, e.g. a vector for encoding or providing the protein function of interest, in which case such treatment method may be for example a gene therapy Inhibition of intracellular protein-protein interactions (ii) may typically include delivery of protein inhibitors into the cell by the use of the ERS peptide. Both methods (i) and (ii) may include the administration of antibodies

In a broad sense gene therapy (iii) is understood broadly herein as providing any nucleic acid into the cell which results in or enhances expression of a gene or which inhibits expression thereof, e.g. results in silencing of said gene like in antisense therapy. For example genes of full length e.g. in the form of vectors can be introduced into the cell by the present methods or the methods include delivery of antisense nucleic acids or regulating nucleic acids like siRNAs or miRNAs. Peptide linkers as well as peptide—nucleic acid linkages are well-known in the art; in preferred version such linkages are cleavable at specific sites. [See e.g. Benizri, S. et al. 2019; Winkler, J. Ther Deliv.]

In case of cancer therapy (iv) the therapeutic method of the invention relates to the administration of an anti-cancer agent which preferably may be a biological molecule, e.g. an anti-cancer protein or an anti-cancer antibody or an anti-cancer therapeutic vaccine, e.g. a peptide vaccine or DNA vaccine. Such solutions are described e.g. in EP3270954A1 and EP3429618A1. In a preferred embodiment delivered are to be killed.

In a further preferred embodiment the therapy is against infected cells (i). Such cell may be cancer cells or cells infected by a pathogenic agent.

The methods according to the invention may include any biological therapy like the administration of antibodies in antibody therapy, cytokines, growth factors, protein inhibitors, enzymes in case of impaired enzyme function, expression factors etc.

In an embodiment the therapy is directed to a condition wherein the cells of the patient express ganglioside GM1, preferably overexpress GM1.

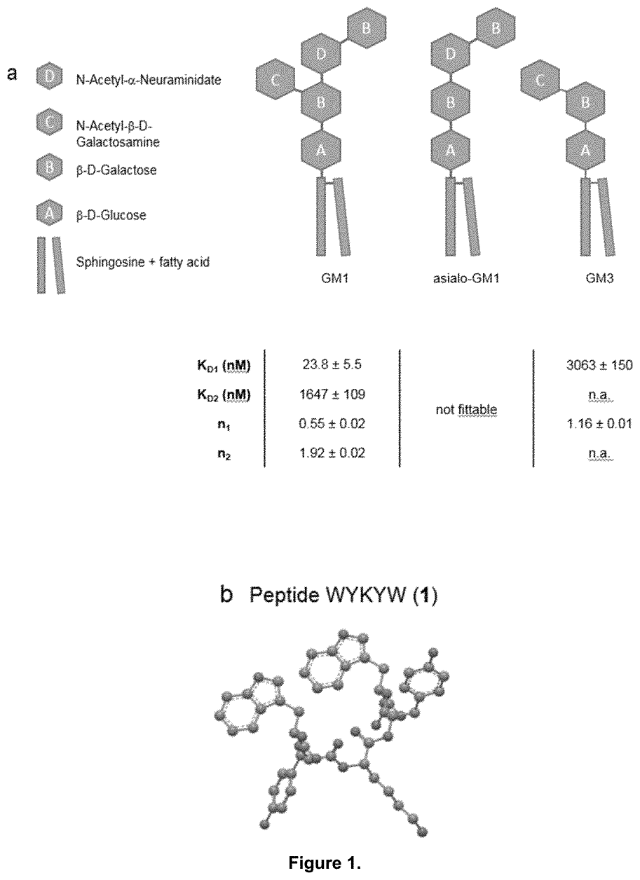

The present inventors have carried out several experiments to exemplify and illustrate to present invention. In particular, the inventors tested several variants of the WYKYW sequence and it has been found that the interaction is highly specific between WYKYW and GM1, which is demonstrated by the affinity data obtained for binding between WYKYW and various ganglioside derivatives (FIG. 1), and for binding between various mutated sequences of WYKYW and GM1 (Table 1). WYKYW and close analogues binds GM1 selectively, the sialyl group and the terminal digalactoside moieties of GM1 are is essential for the high affinity interaction.

It is important to underline that all Ala point mutations and single D-α-amino acid replacements in WYKYW are detrimental for high-affinity binding of GM1, which supports that WYKYW is the minimal motif to be conserved for the high affinity molecular recognition and its stereochemical arrangement is the best for GM1 recognition. The sequence WYKYW is very sensitive to homolog mutations, K3->R3 replacement also destroyed the high-affinity interaction.

However, the present inventors have found that backbone homologation by α->homo-β³ amino acid mutations yielded three sequences with affinities to GM1 below 200 nM. These β-mutations improve the peptidase/protease resistance at the same time, so they also can be regarded as advantageous embodiments of the invention.

Further aspect of the invention is that ERS WYKYW and its high affinity derivatives can be attached to linkers (e.g. PEG based linkers) on the N- or C-terminals without loosing high affinity binding (Table 2).

The constructs can be attached to proteins (e.g., Neutravidin) and the affinitiy to GM1 is retained (see Table 2). This behavior is essential for the ability of routing the cargo to the lipid rafts as specified above.

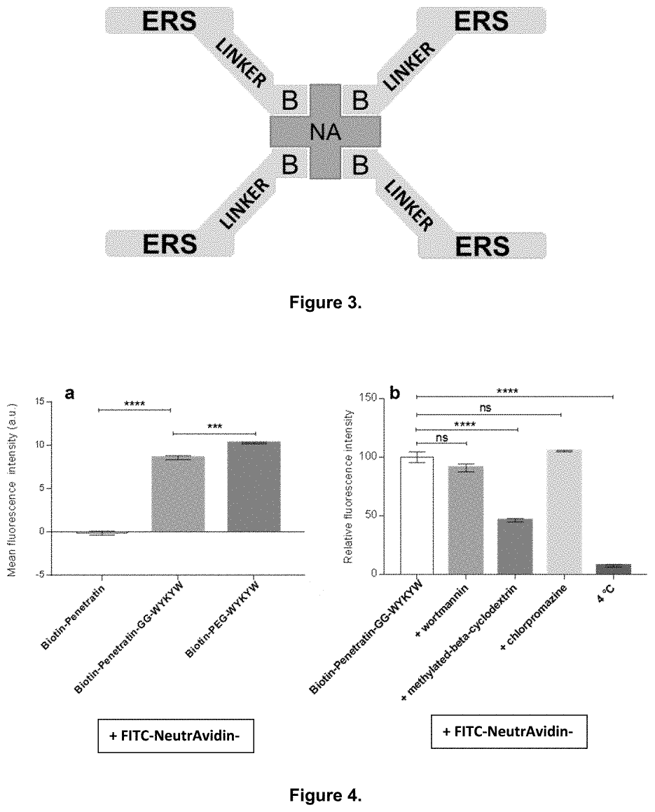

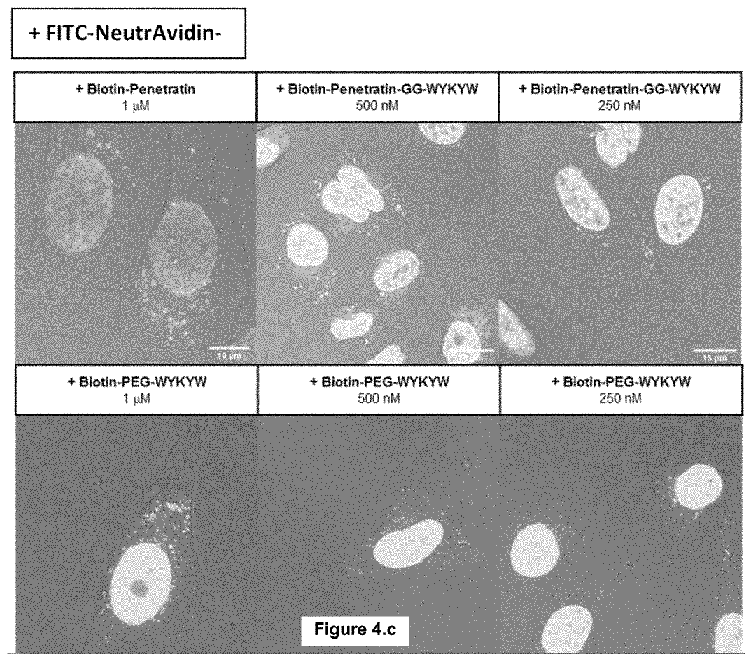

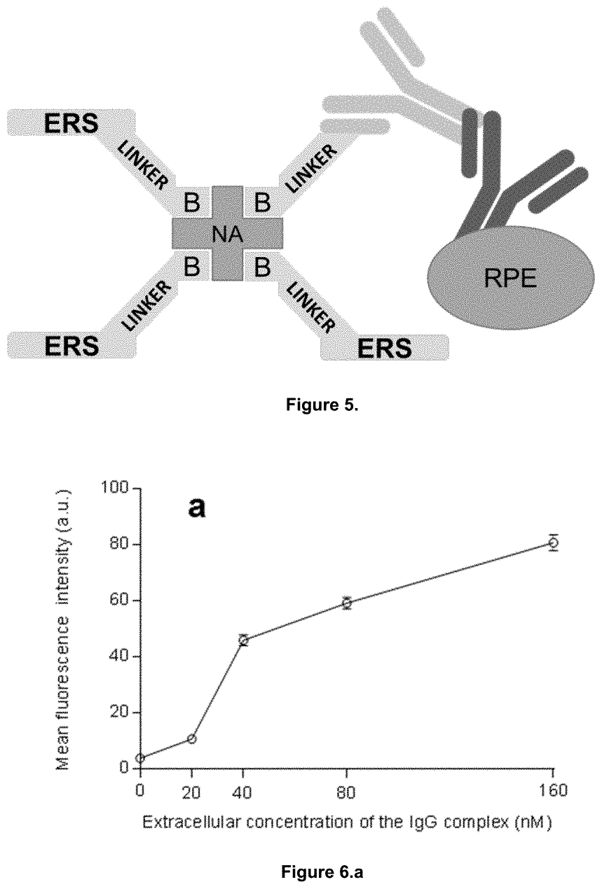

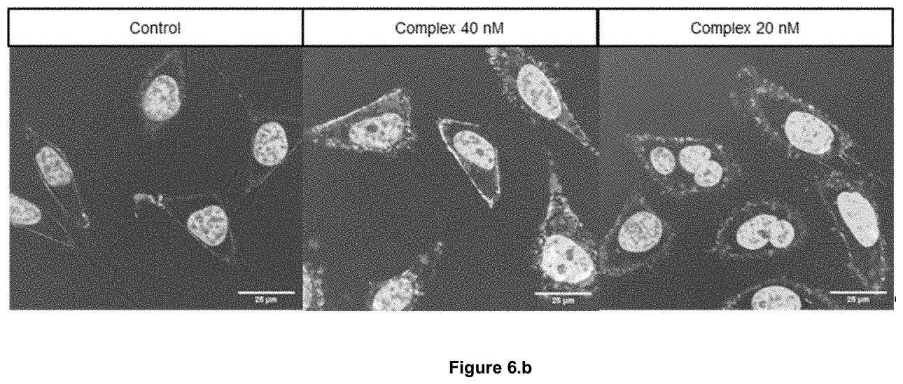

Further aspect of the invention is that the ability of the ERS WYKYW and its high affinity derivatives to route toward and trigger lipid raft-mediated/caveolar endocytosis is retained when attached to a macromolecular cargo. Embodiments of the invention is when WYKYW is linked to Penetratin or PEG-based oligomeric moiety, and these chimerae are attached to Neutravidin (FIG. 3). This was tested at a concentration of 1 μM and below, where cell delivery of the cargo by WYKYW was observed. While Penetratin itself was unable to translocate the protein, constructs with both Penetratin and the ERS peptide WYKYW successfully routed the cargo into the cytosol (FIG. 4). The cell delivery experiments on this model was tested with endocytosis inhibitors, which clearly demonstrated that the mechanism is solely lipid raft-mediated. These findings clearly demonstrate that WYKYW affords selective endocytosis routing of a protein cargo at submicromolar concentrations.

Additional aspect of the invention is that the cargo attached covalently or non-covalently to the ERS WYKYW and its high affinity derivatives avoids the lysosomal degradation (FIG. 4c).

Another aspect of the invention relates to the conjugates of the ERS WYKYW and its high affinity derivatives with cargo, where selective endocytosis routing is carried out at low nanomolar concentrations, which is relevant from therapeutic point of view. An embodiment of the invention is a biomolecular associate including macromolecules (e.g. immunoglubulin G), which is tagged with WYKYW (FIG. 5). Said tagged cargo display intracellular delivery at the concentration range 20-160 nM (FIG. 6)

Further aspect of the invention is that the cargo is not degraded and diffusely distributed in the cytosol at the concentration range 40-160 nM after cell delivery, which is facilitated by the ERS WYKYW and its high affinity derivatives as covalently on non-covalently linked tags. This is demonstrated by the intense fluorescence of the r-phycoerythrin as part of the cargo (FIG. 6).

ERS sequences related to this invention can be synthesized through solid state peptide synthesis with Fmoc-chemistry. An embodiment of the invention when peptide amides were synthesized on Tentagel R RAM resin with (7-azabenzotriazol1-yl)tetramethyluronium hexafluorophosphate as a coupling agent (HATU). Further details can be found in the General Methods part.

Any peptide of the invention, e.g. the ERS-peptide or a peptide conjugate, e.g. a linker-ERS-peptide conjugate of the invention may be utilized or used as a separate molecule, typically as a starting material to prepare a conjugate. Thus, any peptide of the invention can be in a free form, i.e. can have a free amino and/or free carboxy end. Moreover, the peptide can be in a functionalized form, e.g. in a protected and/or activated form as known in peptide chemistry. As to the carboxy end of the peptide it may be e.g. in an amide form.

The peptide also can be protected against proteolysis by using amide bond analoges. Such technique is well known in the art and is reviewed and described e.g. in Avan I et al. [Avan I et al. 2014] and more recent techniques and methods are disclosed and reviewed e.g. by Werner, Halina M. [Werner, Halina M. 2016]. Further stabilization and design of structure may be achieved by alpha, alpha-disubstituted alpha-amino acids comprising foldamers [Oba, Makoto et al. 2019].

The “cargo molecule” designates herewith the molecule, linked to an ESR peptide directly or indirectly, e.g. via linker and/or via receptor-ligand interaction, and e.g. by covalent or non-covalent binding, the cellular internalization of which is facilitated or enabled by the presence of said ESR. In the present invention, “cargo molecules” theoretically includes peptides, proteins, polysaccharides, lipids, combinations thereof including lipoproteins and glycolipids, nucleic acids (e.g. DNA, siRNA, shRNA, antisense oligonucleotides, decoy DNA, plasmid), small molecule drugs (e.g. cyclosporine A, paclitaxel, doxorubicin, methotrexate, 5-aminolevulinic acid), imaging agents (e.g. fluorophore, quantum dots (QDs), radioactive tracers, metal chelates such as gadolinium (Gd3+) low-molecular-weight chelates, superparamagnetic iron oxide (SPIO)) and nanoparticles including liposomes and polimersomes. It is understood that, when the cargo molecule is a peptide, polypeptide or protein, it can comprise one or more peptides, polypeptides or proteins linked together. Also, when the cargo molecule is a nucleic acid, said nucleic acid can comprise one or more nucleic acids where each one encodes one peptide or polypeptide. Also the cargo molecule can be a combination of a protein, a lipid, and/or a polysaccharide including lipoproteins and glycolipids.

The invention is further illustrated below by non-limiting examples. The skilled person is aware that based on the examples alternative embodiments or variants thereof may also fulfil the object of the invention

### EXAMPLES

### Example 1—General Methods

Peptide Synthesis and Purification

Peptide amides were synthesized on Tentagel R RAM resin with (7-azabenzotriazoll-yl)tetramethyluronium hexafluorophosphate as a coupling agent (HATU). Couplings were performed at room temperature with 3 equivalent amino acid excess for 3 hours. Carboxy-fluorescein (CFS) was coupled to the N-terminal of the sequence with the same method. Peptides were cleaved with TFA/water/d,1-dithiothreitol/triisopropylsilane (90:5:2.5:2.5), and then precipitated in ice-cold diethyl ether. The resin was washed with acetic acid and water, and subsequently filtered and lyophilized. Peptides were purified by RP-HPLC on a C18 column. The HPLC eluents were (A) 0.1% TFA in water and (B) 0.1% TFA in ACN. Their purity was confirmed by analytical RP-HPLC and ESI-MS measurements.

Isothermal Titration Calorimetry

Isothermal calorimetric titrations (ITC) were performed with a MicroCal VP-ITC microcalorimeter in pH 7.2 phosphate buffer solution. In individual titrations, 15 μL GM1:DPC 1:5 containing solution was injected from the computer-controlled 300 mL microsyringe at intervals of 300 s into the ligand solution, dissolved in the same buffer as the GM1. All measurements were carried out at 35° C. The ligands concentration in the cell was 15 μM, and the concentration of GM1 in the syringe was 300 μM. Control experiments were performed by injecting GM1 into the cell containing buffer but no ligands Experiments were repeated twice. The experimental data were fitted to the one binding site or two independent sites model (adjustable parameters: ΔHb1, Kd1, n1 and ΔHb2, Kd2, n2) using a nonlinear least-squares procedure. Errors were calculated by jackknife resampling.

Cell Culture

HeLa cells were cultured in advanced MEM (Gibco™, Life Technologies) supplemented with 10% fetal bovine serum (FBS, PAN Biotech), and JN2B4D Jurkat cells were cultured in RPMI-1640 medium (Gibco™) supplemented with 5% FBS. Both media contained penicillin-streptomycin (100 U/mL, Gibco™), and 2 mM L-glutamine (Gibco™) also. Cells were grown in a humidified incubator with 5% CO2 at 37 C.

Preparation of ERS-Peptide—Protein Complexes

To prepare peptide-NeutrAvidin complexes a solution of biotinylated peptides were incubated with FITC-NeutrAvidin in cell culture media with a molar ratio of 4:1. The solution of complexes were added to HeLa cells at different concentrations. For the preparation of antibody (IgG) complexes, a solution of biotinylated peptides were mixed with biotinylated monoclonal mouse anti-human galectin-1 (2c1/6) antibody and unlabeled NeutrAvidin at the molar ratio of 3:1:1, then secondary r-phycoerythrin-conjugated goat anti-mouse IgG (F-(ab′)2, DakoCytomation) antibody was added to the solution at a molar ratio of 1:1 primary and secondary antibody. HeLa cells were incubated with this complex at different concentrations.

Preparation of ERS-Peptide Conjugates Containing a PEG-Based Linker

Peptide amides comprising PEG-based oligomeric moiety were synthesized on Tentagel R RAM resin with (7-azabenzotriazoll-yl)tetramethyluronium hexafluorophosphate as a coupling agent (HATU). Couplings were performed at room temperature with 3 equivalent amino acid excess for 3 hours. To add a PEG-based oligomeric moiety, Fmoc-Ebes (Fmoc-(8-amino-3,6-dioxa-octyl)succinamic acid) was coupled consecutively thrice after the ERS sequence. Peptides were cleaved with TFA/water/d,1-dithiothreitol/triisopropylsilane (90:5:2.5:2.5), and then precipitated in ice-cold diethyl ether. The resin was washed with acetic acid and water, and subsequently filtered and lyophilized. Peptides were purified by RP-HPLC on a C18 column. The HPLC eluents were (A) 0.1% TFA in water and (B) 0.1% TFA in ACN. Their purity was confirmed by analytical RP-HPLC and ESI-MS measurements.

Flow Cytometry

The internalization of peptides and peptide-NeutrAvidin complexes were determined by flow cytometric analysis. Cells (6×10⁴ in 24-well plates) were grown at 37° C. for 24 h. After removal of the medium, cells were washed with PBS and incubated with the peptides or peptide complexes in MEM+1% FBS at 37° C. These cells were then washed with PBS, harvested from the plates with 0.05% trypsin-EDTA, and washed with PBS. Trypanblue (Reanal) and propidium iodide (Fluka) was added to the cells at final concentration of 0.1% and 15 μM, respectively, in PBS immediately before being subjected to flow cytometric analysis (FACSCalibur flow cytometer, BD Biosciences) and data were evaluated using FlowJo™ software (FlowJo, LLC). The fluorescence intensity of control cells and the FITC-NeutrAvidin were subtracted from the intensity of the peptides or peptide-NeutrAvidin complexes. For the ganglioside GM1 content measurement, HeLa and Jurkat cells were incubated on ice for 30 min with 8 μM FITC-choleratoxin B subunit, then subjected to flow cytometric analysis as described above. For the in vitro pull-down assay, Jurkat cells were treated with 1 μM peptides alone, or with 1, 5 and 10 μM galectin-1. For the endocytosis inhibition experiments HeLa cells were preincubated at 37° C. with 5 mg/mL methyl-β-cyclodextrin (MBCD) for 60 min, 10 μM wortmannin for 60 min, or 10 μg/mL chlorpromazine for 30 min. Cells were then incubated with 1 μM peptide—NeutrAvidin complexes 37° C. for 60 min, treated with trypan blue, and used for flow cytometric analysis as described above. All experiments were carried out in triplicate.

Live Confocal Laser Scanning Microscopy

HeLa cells were plated for overnight culturing in MEM+10% FBS at 1.25×104 cells per cm2 (or 1.5×104 cells per channel) on 6-chamber μ-Slide VI 0.4 (IBIDI). Cells were washed with PBS and incubated with the peptide-complexes in MEM+1% FBS medium at different concentrations for different incubation times at 37° C. The cells then were washed with PBS, and in the cases of antibody-complex washing with 100 mM β-lactose (Sigma-Aldrich) and the biotinylated peptide-NeutrAvidin complex without the primary and secondary antibody followed, to remove surface bound complexes. Cells were stained with 100 ng/mL Hoechst 33342 (Sigma-Aldrich) in MEM medium for 30 min at 37° C. In some experiments, after Hoechst staining cells were labeled with LysoTracker Red (Life Technologies) at 75 nM concentration for 30 min at 37° C. according to the manufacterers' instructions. In case of the IgG complex measurements, cell membranes were visualized with a 5 minute treatment of FITC labeled WGA lectin at 0.2 μg/ml at room temperature after the incubation with the complex. Cells were incubated in Leibovitz's L-15 medium (Life Technologies) during microscopic analysis. FITC-NeutrAvidin complexes were treated with 0.1% trypanblue to quench extracellular fluorescence. To observe the localization of the cargo, cell fluorescence was analyzed with Leica SP5 AOBS confocal laser scanning microscope, using the 405 nm UV diode (for Hoechst staining), 488 nm argon laser line (for FITC staining) and 543 nm HeNe laser line (for r-phycoerythrin staining) For emission detection, the appropriate spectral filter was used for each channel.

Image Analysis

To identify cells and extract their properties we used maskRCNN a deep learning-based image segmentation platform and the CellProfiler software for feature extraction. First, cell nuclei were identified based on the Hoechst signal using a very heavily augmented training set of The Data Science Bowl 2018 competition. The augmentation was done by learning image styles and generating synthetic images of similar types with Pix2pix a GAN (generative adversarial network) deep network. A maskRCNN network was then trained and individual nuclei were inferred. The cytoplasm was approximated with a Watershed region propagation algorithm on the FITC-WGA lectin channel. Using the detected objects (nucleus and cytoplasm) as masks, cellular features such as r-phycoerythrin intensity values, textural properties, and morphological descriptors were extracted. For the final statistical analysis the integrated intensities of individual cells were used.

Cytotoxicity Assay

Kinetics of cell reaction to 7 and 9 peptide treatment was monitored by impedance measurement at 10 kHz (RTCA-SP instrument, ACEA Biosciences, San Diego, Calif., USA). Impedance measurement is non-invasive, label-five and real time, and linearly correlates with adherence, growth, number and viability of cells. For background measurements 50 μL cell culture medium was added to the wells, then HeLa cells were seeded at a density of 6×103 cells/well on non-coated 96-well plates with integrated gold electrodes (E-plate 96, ACEA Biosciences). Cells were treated with peptide solutions at 0.1, 0.5, 1, 5 and 10 μM concentrations at the beginning of the plateau phase of growth and the effects were followed for 24 hours. Triton X-100 detergent (1 mg/mL) was used as a reference compound to induce cell death. Cell index was defined as Rn-Rb at each time point of measurement, where Rn is the cell-electrode impedance of the well when it contains cells and Rb is the background impedance of the well with the medium alone.

### Example 2—Binding Between WYKYW and GM1

Binding of Trp-Tyr-Lys-Tyr-Trp (WYKYW) and analogues to various ganglioside derivatives was tested as described under “Isothermal Titration calorimetry” to explore the specificity of the interaction between WYKYW and GM1. Affinity data for binding between WYKYW and various ganglioside derivatives (FIG. 1), and for binding between various mutated sequences of WYKYW and GM1 (Table 1) were obtained by Isothermal Titration calorimetry as described in Example 1 (General methods).

It has been found that WYKYW binds GM1 selectively, whereas the sialyl group and the terminal digalactoside moieties of GM1 are essential for the high affinity interaction (FIG. 1.a). Non-linear least squares fitting against the two independent- and the single binding site models for GM1 and GM3, respectively, have suggested two dissociation steps for GM1 with different stochiometries and a weak binding to GM3 with a 1:1 stochiometry (see Example 1, General methods). For asialo-GM1, ITC enthalpogram did not exhibit fittable features. It has also been shown that the strongest binding (K_{D1}=23.8±5.5) occurs when, as to the stochiometry, one WYKYW sequence binds to two GM1 molecule.

TABLE 1
Binding affinities (K_{D}) and stoichiometries (n) measured
by ITC for the interactions of the mutated sequences with
ganglioside GM1. In the table single D-α-amino acid replacements
are indicated by lower case letters and α −> homo-
β³ amino acid mutations are shown by a lower case “b”.

| | n1 | n2 | K_{D1}/nM | K_{D2}/nM |
|---|---|---|---|---|
| WYKYW | 0.55 | 1.92 | 23.8 | 1647 |

| | |
|---|---|
| AYKYW | not fittable |

| | | | | |
|---|---|---|---|---|
| WAKYW | 1.0 | — | 5755 | — |
| WYAYW | 1.3 | — | 10467 | — |
| WYKAW | 1.0 | — | 1694 | — |
| WYKYA | 1.0 | — | 1060 | — |
| wYKYW | 1.81 | — | 881 | — |
| WyKYW | 2.05 | — | 892 | — |
| WYkYW | 1.86 | | 4522 | |
| WYKyW | 0.93 | | 3243 | |
| WYKYw | 1.24 | | 3926 | |
| bWYKYW | 0.52 | 1.85 | 4.3 | 2100 |
| WbYKYW | 0.51 | 1.72 | 60 | 1000 |
| WYbKYW | 0.50 | 1.93 | 332 | 1500 |
| WYKbYW | 0.49 | 1.80 | 40.0 | 1200 |
| WYKYbW | 0.50 | 1.76 | 3940 | 3976 |

The binding experiment also has shown that all Ala point mutations and single D-α-amino acid replacements (lower case letters) in WYKYW are detrimental for high-affinity binding of GM1 (Table 1). The sequence WYKYW is very sensitive to homolog mutations, K3->R3 replacement also destroyed the high-affinity interaction. On the other hand, scanning the sequence with backbone homologation by α->homo-β³ amino acid mutations yielded three sequences with affinities to GM1 below 200 nM. These β-mutations improve the peptidase/protease resistance at the same time.

Thus the WYKYW sequence appears to be highly conserved.

### Example 3—Preparation and Study of Peptide and Protein Constructs

ERS WYKYW was attached to linkers on the N- or C-terminals (Table 2). In a set of experiments, the constructs were also labelled e.g. by carboxyfluorescein (CFU). Preparation of these constructs was carried out as described in Methods (Peptide synthesis and purification).

TABLE 2
Binding affinities (K_{D}) and stoichiometries (n) measured by ITC for the interactions
of the designed sequences with GM1. CFU stands for carboxyfluorescein tag.

| Sequence | K_{D1} (nM) | K_{D2} (nM) | n₁ | n₂ |
|---|---|---|---|---|
| WYKYW | 23.8 ± 5.5 | 1647 ± 109 | 0.55 ± 0.02 | 1.92 ± 0.02 |
| CFU-WYKYW | 3420 ± 82 | n.a | 2.08 ± 0.11 | n.a |
| CFU-Penetratin | n.a | | | |
| WYKYW-GG-Penetratin | 37.6 ± 6.8 | 1376 ± 133 | 0.45 ± 0.02 | 2.13 ± 0.04 |
| Penetratin-GG-WYKYW | 45.2 ± 11 | 386 ± 63 | 0.45 ± 0.09 | 2.12 ± 0.11 |
| CFU-WYKYW-GG-Penetratin | 1511 ± 324 | n.a. | 0.48 ± 0.05 | n.a. |
| CFU-Penetratin-GG-WYKYW | 141 ± 45 | 3526 ± 497 | 0.47 ± 0.02 | 1.98 ± 0.03 |
| (Biotin-PEG-WYKYW)₄NeutrAvidin | 14.5 ± 1.7 | 128 ± 5.7 | 0.25 ± 0.01 | 2.21 ± 0.01 |
| (Biotin-Penetratin-GG-WYKYW)₄NeutrAvidin | 20.8 ± 2.4 | 829 ± 37 | 0.25 ± 0.01 | 1.93 ± 0.02 |

The ERS peptide WYKYW showed a high affinity to GM1. Direct coupling of CFU to WYKYW was detrimental to the affinity to GM1.

However, ERS WYKYW was able to retain its nanomolar affinity to GM1 when attached to various linkers (Table 2). For example the linker GG-Penetratin and Penetratin-GG coupled to the C-terminal or to the N-terminal of the WYKYW sequence both maintained binding affinity to GM1 in the nanomolar range. However, the binding affinity of the former construct was significantly reduced by coupling CFU to the N-terminal of WYKYV-Penetratin-GG, whereas was largely maintained by coupling CFU to the N-terminal of Penetratin-GG-WYKYV.

Thus, appropriately selected and positioned linker sequences are appropriate to maintain the high affinity WYKYW-GM1 contact.

It was found that WYKYW triggered the process of endocytosis in human cells. Moreover, when the sequence WYKYW or its analogue was attached to Penetratin, the cell penetration efficiency of Penetratin is highly increased at 1 μM, a magnitude below a concentration where Penetratin is normally applied (10 μM, HeLa and Jurkat cells) (FIGS. 2a and 2b, respectively) indicating that WYKYW opens an additional pathway for cell delivery.

WYKYW is not toxic even high concentrations (10 μM), which is well above its effective concentration needed for cell delivery. (FIG. 2c).

WYKYW was also attached to NeutrAvidin through linkers penetratin or PEG-based oligomer (scheme the general representation on FIG. 3). Solution of biotinylated ERSs were incubated with FITC-NeutrAvidin in cell culture media with a molar ratio of 4:1 as described in Example 1 (General methods, Preparation of ERS-protein complexes). These materials was tested on HeLa and Jurkat cells by using flow cytometry and confocal microscopy as described Example 1 (General methods, Flow cytometry and Live confocal laser scanning microscopy).

Experiments were carried out at a concentration of 1 μM and below, where cell delivery of the cargo by WYKYW was observed, but Penetratin in itself was unable to translocate the protein (FIG. 4.a). Cell delivery was observed when WYKYW was attached to the protein either through Penetratin-GG linker or through a PEG-based linker (FIG. 4.a). These findings underline that ERS WYKYW is the essential component for the effect, while penetratin and a PEG-based linker act only as linkers at this concentration. The constructs comprising a PEG-based oligomeric moiety have not contained the GG spacer as this moiety has a spacer role as well.

The cell delivery experiments on this model was tested with endocytosis inhibitors, which clearly demonstrated that the mechanism is solely lipid raft-mediated, as wortmannin inhibited the uptake of the Biotin-Penetratin-GG-WYKYW construct complexed by FITZ-NeutrAvidin, whereas other endocytosis inhibitors like methylated β-cyclodextrin and chlorpromazine not. These findings clearly demonstrate that WYKYW affords selective endocytosis routing of a protein cargo at submicromolar concentrations.

In further cellular uptake experiments by HeLa cells, staining of the constructs Biotin-Penetratin, Biotin-Penetratin-GG-WYKYW and Biotin-PEG-WYKYW, cell nuclei and lysosomes and by using confocal laser scanning microscopy images of HeLa cells that the mechanism is different from the progression of the internalized caveolae to endosomes and later to lysosomes. It was found that the cargo attached covalently or non-covalently to the ERS WYKYW and its high affinity derivatives avoids the lysosomal degradation (FIG. 4c). In case of the constructs containing PEG-based linkers no lysosome staining was applied (FIG. 4c, lower panels)

### Example 4—Preparation and Study of IgG Containing Protein Constructs

In a further experiment a biomolecular associate including macromolecules (e.g. immunoglubulin G), which is tagged with WYKYW (FIG. 5). The biotynilated ERSs were prepared and tested as described in Example 1 (General methods; Preparation of ERS-protein complexes, Live confocal laser scanning microscopy and Image Analysis).

A solution of biotinylated ERSs were mixed with biotinylated monoclonal mouse anti-human galectin-1 (2c1/6) antibody and unlabeled NeutrAvidin at the molar ratio of 3:1:1, then secondary r-phycoerythrin-conjugated goat anti-mouse IgG (F-(ab′)2, DakoCytomation) antibody was added to the solution at a molar ratio of 1:1 primary and secondary antibody. Said tagged cargo display intracellular delivery at the concentration range 20-160 nM (FIG. 6a). The amount of the delivered material is dependent on the extracellular concentration applied, and it tapers off at around 20 nM, which is the affinity limit determined for the WKYW-GM1 interaction (see Table 1).

The IgG-containing cargo was not degraded and diffusely distributed in the cytosol at the concentration range 40-160 nM after cell delivery, which is facilitated by the ERS WYKYW and its high affinity derivatives as covalently on non-covalently linked tags. This is demonstrated by the intense fluorescence of the r-phycoerythrin as part of the cargo (FIG. 6b).

Summary Thus, the construct is suitable for cell delivery of large cargos including noncovalent associates of biomolecules without cytotoxicity in the nanomolar range which is therapeutically relevant.

### INDUSTRIAL APPLICABILITY

Active ingredients which are difficult to be transported inside a cell can be easily transported there by using ESRs according to the present invention. This means that intracellular targets previously beyond the reach of the existing cell delivery technologies at nanomolar extracellular concentrations can be targeted. Moreover, efficacy of active ingredients can be increased and therefore the dosage can be lowered. As a result, side effects due to a drug administration can be minimized and effectiveness of treatment can be increased.

REFERENCES

Akishiba, M., T. Takeuchi, Y. Kawaguchi, K. Sakamoto, H.-H. Yu, I. Nakase, T. Takatani-Nakase, F. Madani,
A. Gräslund and S. Futaki, Nature chemistry, 2017, 9, 751.
André, S., C. E. P. Maljaars, K. M. Halkes, H.-J. Gabius and J. P. Kamerling, Bioorganic & medicinal
chemistry letters, 2007, 17, 793-798.
Andre, S., C. J. Arnusch, I. Kuwabara, R. Russwurm, H. Kaltner, H. J. Gabius and R. J. Pieters, Bioorganic &
medicinal chemistry, 2005, 13, 563-573.
Avan I et al. Peptidomimetics via modifications of amino acids and peptide bonds. Chem Soc Rev. 2014 May
21; 43(10): 3575-94. doi: 10.1039/c3cs60384a
Benizri, S. et al. Bioconjug Chem. 2019 Feb 20; 30(2): 366-383.;
De Jong, E., D. S. Williams, L. K. Abdelmohsen, J. C. Van Hest and I. S. Zuhorn, Journal of Controlled
Release, 2018, 289, 14-22.
El-Andaloussi, S., P. Jarver, H. J. Johansson and U. Langel, Biochem J, 2007, 407, 285-292.
Erazo-Oliveras, A., K. Najjar, L. Dayani, T.-Y. Wang, G. A. Johnson and J.-P. Pellois, Nature methods, 2014,
11, 861.
Fajka-Boja, R., A. Blasko, F. Kovacs-Solyom, G. Szebeni, G. Toth and E. Monostori, Cellular and molecular
life sciences, 2008, 65, 2586-2593.
Fischer, S. K., J. Yang, B. Anand, K. Cowan, R. Hendricks, J. Li, G. Nakamura and A. Song, mAbs, 2012, 4,
623-631.
Fosgerau, K. and T. Hoffmann, Drug Discovery Today, 2015, 20, 122-128.
Fuster, M. M. and J. D. Esko, Nature Reviews Cancer, 2005, 5, 526.
Gautam, A., H. Singh, A. Tyagi, K. Chaudhary, R. Kumar, P. Kapoor and G. P. S. Raghava, Database, 2012,
2012, bas015-bas015.
Journal of Cell Science (2016) 129, 2473-2474
Kauffman, W. B., Fuselier, T., He, J., & Wimley, W. C. 2015, 40(12), 749-764.
Kiss, A. L. and E. Botos, Journal of cellular and molecular medicine, 2009, 13, 1228-1237.
Kopitz, J., C. von Reitzenstein, M. Burchert, M. Cantz and H.-J. Gabius, Journal of Biological Chemistry,
1998, 273, 11205-11211.
Krengel, U. and P. A. Bousquet, Frontiers in immunology, 2014, 5, 325.
Landon, L. A., Zou, J., & Deutscher, S. L. Molecular diversity, 2004, 8(1), 35-50.
Lättig-Tünnemann, G., M. Prinz, D. Hoffmann, J. Behlke, C. Palm-Apergi, I. Morano, H. D. Herce and M. C.
Cardoso, Nature communications, 2011, 2, 453.
LeCher, J. C., Nowak, S. J., & McMurry, J. L. Biomolecular concepts, 2017, 8(3-4), 131-141.
Maljaars, C. E. P., S. André, K. M. Halkes, H.-J. Gabius and J. P. Kamerling, Analytical biochemistry, 2008,
378, 190-196.
Matsubara, T., Ishikawa, D., Taki, T., Okahata, Y., & Sato, T. FEBS letters, 1999, 456(2), 253-256.
Matsubara, T., Iijima, K., Nakamura, M., Taki, T., Okahata, Y., & Sato, T. Langmuir, 2007, 23(2), 708-714.
Matsubara, T., R. Otani, M. Yamashita, H. Maeno, H. Nodono and T. Sato, Biomacromolecules, 2017, 18, 355-362.
McNaughton, B. R., J. J. Cronican, D. B. Thompson and D. R. Liu, Proceedings of the National Academy of
Sciences, 2009, 106, 6111-6116.
Montesano, R., J. Roth, A. Robert and L. Orci, Nature, 1982, 296, 651-653.
Oba, Makoto et al. Secondary structures and cell-penetrating abilities of arginine-rich peptide foldamers
Scientific Reportsvolume 9, Article number: 1349 (2019)
Pelkmans, L. and A. Helenius, Traffic, 2002, 3(5), 311-320.
Pelkmans, L., J. Kartenbeck and A. Helenius, Nat Cell Biol, 2001, 3, 473-483.
Pelkmans, L., T. Burli, M. Zerial and A. Helenius, Cell, 2004, 118, 767-780.
Qian, Z., J. R. LaRochelle, B. Jiang, W. Lian, R. L. Hard, N. G. Seiner, R. Luechapanichkul, A. M. Barrios and
D. Pei, Biochemistry, 2014, 53, 4034-4046.
Saar, K., M. Lindgren, M. Hansen, E. Eiríksdóttir, Y. Jiang, K. Rosenthal-Aizman, M. Sassian and Ü. Langel,
Analytical Biochemistry, 2005, 345, 55-65.
Sánchez-Navarro, M., M. Teixidó and E. Giralt, Nature chemistry, 2017, 9, 727.
Smith, A. E. and A. Helenius, Science, 2004, 304, 237-242.
Walensky, L. D., A. L. Kung, I. Escher, T. J. Malia, S. Barbuto, R. D. Wright, G. Wagner, G. L. Verdine and S.
J. Korsmeyer, Science, 2004, 305, 1466-1470.
Wéber, E., A. Hetényi, B. Váczi, E. Szolnoki, R. Fajka-Boja, V. Tubak, É. Monostori and T. A. Martinek,
Chembiochem, 2010, 11, 228-234.
Werner, Halina M., Peptide Backbone Composition and Protease Susceptibility: Impact of Modification Type,
Position, and Tandem Substitution. Chembiochem. 2016 Apr 15; 17(8): 712-718
Winkler, J. Ther Deliv. 2013 Jul; 4(7): 791-809.
Zorko, M. and Ü. Langel, Advanced Drug Delivery Reviews, 2005, 57, 529-545.
Zuris, J. A., D. B. Thompson, Y. Shu, J. P. Guilinger, J. L. Bessen, J. H. Hu, M. L. Maeder, J. K. Joung, Z.-Y.
Chen and D. R. Liu, Nature biotechnology, 2015, 33, 73.
Zuris, J. A., D. B. Thompson, Y. Shu, J. P. Guilinger, J. L. Bessen, J. H. Hu, M. L. Maeder, J. K. Joung, Z.-Y.
Chen and D. R. Liu, Nature biotechnology, 2015, 33, 73.

1. A method for mediating the delivery of a cargo into cells via ganglioside binding triggering lipid-raft mediated endocytosis,
said method comprising a non-therapeutic use of a peptide of general formula R1-R2-Lys-R3-Trp,
wherein R1 is Trp or b³-homo-Trp, R2 is Tyr or b³-homo-Tyr, and R3 is Tyr or b³-homo-Tyr,
as an endocytosis routing sequence peptide (ERS peptide), by conjugating said cargo to said peptide.

2. The method according to claim 1, wherein said cargo is selected from the group containing biologically active molecules, diagnostic molecules and nanoparticles.

3. The method according to claim 1, wherein the peptide is the Trp-Tyr-Lys-Tyr-Trp.

4. A conjugate of a peptide of general formula R1-R2-Lys-R3-Trp, wherein R1 is selected from Trp and β³-homo-Trp, R2 is selected from Tyr and β³-homo-Tyr R3, and R3 is selected from Tyr and β³-homo-Tyr, said conjugate comprising
said peptide as an endocytosis routing sequence peptide (ERS peptide),
a cargo, and
optionally a linker moiety between said peptide and cargo.

5. The conjugate according to claim 4 wherein said cargo is selected from the group containing biologically active molecules, diagnostic molecules and nanoparticles.

6. The conjugate according to claim 4, where the ERS peptide and the biologically active molecule are bound together via a linker, preferably coupled to the N-terminus of the said endocytosis routing sequence, which is selected form the group containing:
a) a spacer sequence, preferably an oligopeptide, more preferably GG,
b) a stabilizing moiety or sequence, preferably an oligopeptide of positive charge, more preferably a penetratin moiety (RQIKIWFQNRRMKWKK) or analogue thereof,
c) a PEG-based oligomeric moiety and
d) any combination of linkers a) to c).

7. The conjugate according to claim 4, where the ERS peptide has the sequence of Trp-Tyr-Lys-Tyr-Trp.

8. The conjugate according to claim 6, wherein the linker is the penetratin molecule (RQIKIWFQNRRMKWKK) coupled to the N-terminus of the said ERS peptide:

9. A polynucleotide encoding a polypeptide suitable for mediating the delivery of a protein cargo into cells via ganglioside binding triggering lipid-raft mediated endocytosis, said polypeptide comprising
a peptide having the sequence of Trp-Tyr-Lys-Tyr-Trp, and
a linker moiety, and/or
a cargo molecule.

10. The conjugate according to claim 4 for use in the therapy of a disease wherein the cargo is a therapeutically active molecule.

11. The conjugate of claim 4 wherein the peptide is selected from the following group:
Trp-Tyr-Lys-β³-homo-Tyr-Trp,
Trp-β³-homo-Tyr-Lys-Tyr-Trp,
Trp-β³-homo-Tyr-Lys-β³-homo-Tyr-Trp,
β³-homo-Trp-Tyr-Lys-Tyr-Trp,
β³-homo-Trp-β³-homo-Tyr-Lys-Tyr-Trp,
β³-homo-Trp-β³-homo-Tyr-Lys-β³-homo-Tyr-Trp,
β³-homo-Trp-Tyr-Lys-β³-homo-Tyr-Trp
and salts, amides and protected forms thereof.
